(19) United States
(12) Patent Application Publication
(10) Pub. No.: US 2024/0248040 A1
(43) Pub. Date: Jul. 25, 2024

(54) IN-LINE EARLY WARNING SYSTEM OF WATER CONTAMINATION WITH ORGANIC MATTER

(71) Applicant: THE STATE OF ISRAEL MINISTRY OF AGRICULTURE & RURAL DEVELOPMENT, AGRICULTURAL RESEARCH ORGANIZATION, Rishon Lezion (IL)

(72) Inventors: Iftach KLAPP, Modiin (IL); Shlomo SELA, Modiin-Maccabim-Reout (IL); Mikhail BORISOVER, Herzliya (IL); Lavi ROSENFELD, Rishon Lezion (IL)

(21) Appl. No.: 18/562,320

(22) PCT Filed: Jun. 7, 2022
(86) PCT No.: PCT/IL2022/050602

§ 371 Date: Nov. 19, 2023

Related U.S. Application Data
(60) Provisional application No. 63/197,525, filed on Jun. 7, 2021.

(51) Int. Cl. G01N 21/64

(52) U.S. Cl. CPC G01N 21/6486; G01N 21/645; G01N 2021/6421; G01N 2021/6471

(57) ABSTRACT

An inline early warning system of water contamination with fluorescence signature comprises: (a) an optical chamber embeddable into a water-supply system and having an internal passage configured for conducting a flow of water to be tested (b) a UV light source configured for illuminating the flow of water via the entrance window and excite tryptophan-like fluorescence at 280 nm and humic-like-fluorescence at 320-360 nm; (c) an optical sensor arrangement configured for sensing the tryptophan-like fluorescence and humic-like-fluorescence emissions at 350 nm and 400 to 480 nm via the exit window, respectively; (d) a UV light source configured for illuminating the flow of water via the entrance window and excite natural fluorescence of water contamination; (e) a non-optical sensor arrangement; (f) an acquisition and control unit configured for measuring tryptophan-like fluorescence and humic-like-fluorescence emissions and (g) a processor configured processing obtained measured data.

### FIELD OF THE INVENTION

The current invention relates to a system for monitoring water quality, biological contamination in water and, more specifically to a fluorescent system based on tryptophan-like-fluorescence and humic-like fluorescence. The invention includes a circulation Jig allowing of simulating field conditions requires for the sake of developing the said system.

### BACKGROUND OF THE INVENTION

Use of a treated wastewater as irrigation water source is a common practice around the globe. This concept allows to reduce demand for water from other sources (e.g., groundwater) and got attention in recent years due to expected water shortage in near future. Therefore, nowadays use of treated wastewater for irrigation is higher than ever. In Israel, for instance, treated water constitutes 38% of overall agricultural water consumption in 2015, in 2019 it was rise to 45%. While using treated water save water resources, this technique not as safe as use of fresh water from natural resources. Typical wastewater contains variety of inorganic substances from domestic and industrial sources, organic matter (OM), and dissolved organic matter (DOM) for the sake of simplicity of terminology, farther on, we refer to both OM and DOM as DOM. Most conventional thereat techniques, like chlorination, not only found to be harmful for human and environment, but also are inefficient. As a result, online water monitors and early warning contamination detectors for treated wastewater lines might play a crucial role in water systems. Fluorescence spectroscopy is a well-known technique for water analyses, and in recent year received additional attention due to availability of light emitting diodes (LED) as UV sources. Analysis of excitation of tryptophan-like fluorescence (TLF) at 280/350 nm emission and humic-like-fluorescence (HLF) emission at 320-360/400-480 nm emission/excitation pairs may reveal OM and DOM contamination respectively. Therefore, numerous methods implement fluorescence spectroscopy for detection of OM and DOM in different environments such as oceanic, urban and others.

Those methods have the same goal, but mostly divided in to two main categories, field-based system that monitors quality of a water reservoir by publicly available portable fluorimeters and custom-built fluorimeter prototypes for controlled environment measurements.

While former works deal with environmental conditions, later aim to build cheap and reliable prototype for specific task under known conditions. In most cases wastewater plant and small-scale irrigation systems left without attention. Moreover, In these systems monitoring rely on a simple excitation/emmission setups which did not aimed to a proper. Special care requires irrigation systems because of large amount of water to process from unknown, inconsistent and time dependent water sources.

Consequently, the proposed method aims to fill those gaps. Presented system successfully monitors fluorescence of thread water for irrigation by examining TLF and HLF signals that occur in range of 300-520 nm, and provide a range of additional measure parameters (e.g. temperature) for later signal processing. While TLF/HLF signals are typical for DOM contamination, upon adjustment of the illumination sources and spectro-flourometer one may adjust the proposed system to detect other contaminations.

We show a real implementation of array passed photo multipliers sensing which both measure flow system and full fluorescence spectra, which for the best of our knowledge suggested for the first time. DOM break into the irrigation water line is demonstrated using, milk, which is rich with proteins that highly fluorescent for 280 nm and 340 nm light source. Moreover, substance flow and sensors are in outdoor condition, thus measurements are affected by weather and additional environmental conditions that encountered in common farm or plant. Realizing the outdoor conditions is essential for sensors developing, testing and data collection. Since bringing the optical lab to the field is impractical, a cycling system which mimics flow in a standard irrigation system is presented, such that the field conditions are simulated with every experiment. In a first embodiment, the measurement technique suits standard flow rate of a 1″ pipe system, and may be modified for different pipe systems, i.e. not limited to cuvette dimensions, to a low flow rate and may be realized to a large diameter pipe system. The test samples for evaluation of the proposed system were taken from irrigation water reservoirs, which consist of mixed treated water and sweet water.

Linear regression problems are very common for modeling chemo-metrics relations between concentrations of chemicals content in material to the measured spectral emission while illuminated by a known light source. For instance, such a method used to estimate and model pollution of water by fuel and biological sources. Notable methods are original least squares (OLS), partial least squares (PLS), support vector regression (SVR). Yet, in many cases spectroscopy dependence on chemical compounds is non-linear problem, where one must or find workaround to force linearity (e.g. kernel trick). Uses of linear regression is feasible in a controlled environment, i.e. in laboratory with precise equipment and control over effect of environment. Data acquired in such a setting is denoted as high-quality data. Moreover, even if a model has desirable results, next step of a standard application for a method may be implementation in field conditions. Under field conditions researcher has lesser control on environment, and measurement equipment produces less accurate samples. In this case data denoted as low-quality data. As result previously trained models which worked well on high-quality data may achieve poor results in this setting.

Establish linear regression on data collected in the field is challenging from few reasons. First model developed on high quality lab measurements, is hardly usable such that training starts from the basic level. Second, there is difficult to use a data measured in the lab which is easier to measured, to enrich the field measurements which are harder to collect. Third when the changing environment in the outdoor effect the interaction of light and matter, linear relation between spectra and concentration which worked well in the lab, changes, resulted in a poor prediction of the trained linear model.

An alternative estimation approach to tackle the regression problem of measurements taken under environmental conditions is to establish a deep neural networks (DNN) model. When DNNs are used there is also a possibility to perform a transfer learning technique, i.e. train model on high-quality data, and then adopt it to low-quality data. Naturally, transfer learning for DNN models preformed on well-known architecture for image classification tasks, where a part of weights are replaced by newly initialized ones and trained with new data.

DNN are abstract mathematical models, composed of enormous parameters which learn hidden properties and features of the data. Many times, the complicated and abstract feature representation is non interpretive for human. Nevertheless, this abstract representation works, and even in more sophisticated tasks such as domain transfer (DT). DT models are based on DNN auto-encoders, thus samples encoded to compact representation, or features, with pre-defined dimension. The DT features are also non interpretive, but it is easier to construct a training process that will guide model to simultaneous mapping of different domains to with the same feature set. Novel DT techniques even allow to control an effect of each feature set on output. Yet, in all of the mentioned techniques correlation between features and physical model found after series of tests, and not straight forward by initial design.

Both of the challenges, namely knowledge transfer from one model to the other, and estimation of true physical model, arise in the very important research field of water quality monitoring by chemo-metrics techniques. Due to high demand for clean water in the world, that will only rise, many works are dedicated to fast and accurate systems for water quality estimation based on analysis of emitted fluorescence spectrum (EFS). Even if prototypes are robust and relatively precise, result of a work limited by estimation technique, which mostly is linear methods with modification based on physical model.

### SUMMARY OF THE PRESENT INVENTION

It is hence one object of the invention to disclose an inline early warning system of water contamination with organic matter or other water quality contamination which may be detected by transmission or flouresence spectroscopy. The aforesaid system comprises: (a) an optical chamber embeddable into a water-supply system; the optical chamber having an internal passage configured for conducting a flow of water to be tested; the optical chamber having optically transparent entrance and exit windows; (b) a UV light source configured for illuminating the flow of water via the entrance window and excite tryptophan-like fluorescence at 280 nm and humic-like-fluorescence at 320-360 nm; (c) an optical sensor arrangement configured for sensing the tryptophan-like fluorescence and humic-like-fluorescence emissions at 350 nm and 400 to 480 nm via the exit window, respectively; (d) a non-optical sensor arrangement; (e) an acquisition and control unit configured for measuring tryptophan-like fluorescence and humic-like-fluorescence emissions and (f) a processor configured processing obtained measured data.

Another object of the invention is to disclose the optical sensor arrangement comprises disclosed along to a propagation path of a fluorescence emission light beam an optical tube, a spectral dispersing element and an optical sensor.

A further object of the invention is to disclose the exit window and optical sensor arrangement optically connected by an optical fiber.

A further object of the invention is to disclose the spectral dispersing element which is a diffraction grating.

A further object of the invention is to disclose the optical sensor which is a photoelectric multiplier tube.

A further object of the invention is to disclose the non-optical sensor which is a photoelectronic multiplier tube.

A further object of the invention is to disclose the UV light source which is a deep ultraviolet LED array.

A further object of the invention is to disclose the arrangement comprising at least one sensor selected from the group consisting of a flow meter, a conductivity meter and thermocouple and any combination thereof.

A further object of the invention is to disclose the processor preprogrammed for performing: (a) a step of single domain training with high quality data; and (b) a new transfer learning step.

A further object of the invention is to disclose the transfer learning step comprising initializing a new encoder and replacing the new encoder with a Siamese encoder.

A further object of the invention is to disclose the circulation Jig allowing of simulating field conditions requires for the sake of developing the said system.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic diagram of an in-line early warning system of water contamination with an organic matter, mounted on the circulation Jig;

FIGS. 2a to 2d are photographs of an in-line early warning system of water contamination with an organic matter, mounted on the circulation Jig;

FIGS. 2e to 2g are cross-sectional and side views of an optical chamber;

FIG. 3 is a schematic diagram of a UV source control arrangement;

FIG. 4 is a schematic diagram of a non-optical sensor arrangement;

FIG. 5 is a schematic diagram of data processing software;

FIG. 6 is a schematic diagram of a pipe system;

FIG. 7 is a schematic diagram of an optical arrange of a spectrofluorometer;

FIG. 8 is a flowchart of a postprocessing procedure of a fluorescence signal;

FIG. 9 is a spectral graph of milk reference fluorescence signal under excitation of 280 nm and 380 nm;

FIGS. 10a and 10b are spectral graphs of fluorescent signal before and after correction, respectively;

FIG. 11 is spectral graph of fluorescent exemplary signals with excitation of 280 nm obtained in laboratory conditions in comparison with signal measured by the in-line early warning system;

FIG. 12 is a graph of maximum value of TLF intensity signal versus temperature value;

FIG. 13 is a spectral graph of milk fluorescence emission under excitation of 280 nm measured by the in-line early warning system;

FIG. 14 is a comparative graph of fluorescence signals measured in irrigated water and various concentrations of milk in the same water;

FIG. 15 is a temporal graph of maximum value of TLF intensity signal and temperature;

FIG. 16 is a temporal graph of bacteria quantity in substance;

FIG. 17 is a spectral graph of fluorescence signals obtained with excitation wavelength 280 nm;

FIG. 17b is a spectral graph of fluorescence signals obtained with excitation wavelength 340 nm;

FIG. 17c is an enlarged spectral graph of fluorescence signals of HLF peak range obtained with excitation wavelength 340 nm;

FIGS. 18a and 18b illustrate effect of milk injection on TLF and HLF; FIG. 14a corresponds to TLF and HLF measured in real time while FIG. 14b to TLF and HLF measured 24 hours later;

FIGS. 19a to 19c illustrate sensory values after smooth for a course of 48 hours; vertical green dashed line indicates milk injections time; FIGS. 15a, 15b and 15c correspond to flow speed measurements, temperature measurements of the waters in the tank and water conductivity, respectively;

FIG. 20 shows spectral graphs of fluorescence under excitation of 280 nm before and after fiber cleaning by wipes;

FIGS. 21a and 21b are exemplary graphs of data series sorted by temperature (FIG. 17a) and concentration (FIG. 17b);

FIG. 22 is a spectral graph of measured fluorescence signals;

FIG. 23 is a spectral graph of fluorescence signals measured in high resolution;

FIG. 24 illustrates visualization of sample space (left) and latent space (right) for trained Siamese network with contrastive loss;

FIGS. 25a and 25b are flowcharts of a training process for single domain; FIG. 21a corresponds to forward pass while FIG. 21b to back propagation of a gradient;

FIG. 26 is a flowchart of a training process for an additional domain;

FIGS. 27a to 27c are regression fit curves for scenario A (d_{HQ}=221); ĉ is an estimated concentration, c is a ground truth; green line marks highest training concentration of 500 ppb;

FIG. 27a corresponds to ER, FIG. 27b to R and FIG. 27c to FC;

FIGS. 28a to 28c are regression fit curves for scenario B (d_{LQ}=32) limited to low concentration values; ĉ is an estimated concentration, c is a ground truth; green line marks highest training concentration of 500 ppb; FIG. 28a corresponds to ER, FIG. 28 to R and FIG. 28c to FC; and

FIGS. 29a and 29b are spectral graphs of typical fluorescence signals of DDW prior to tryptophan injection for each series. before base line reduction and after baseline reduction, respectively.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide an in-line early warning system of water contamination with organic matter.

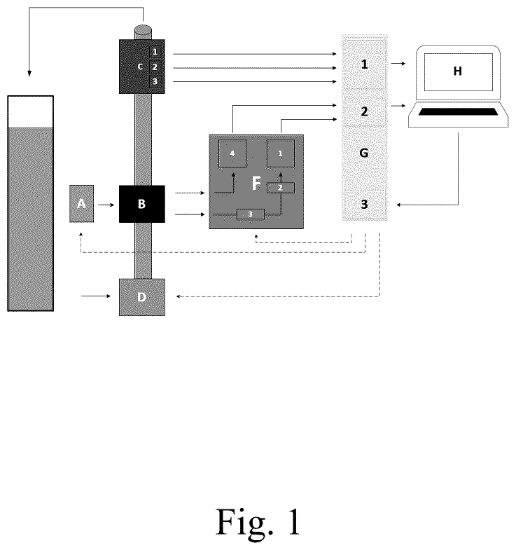

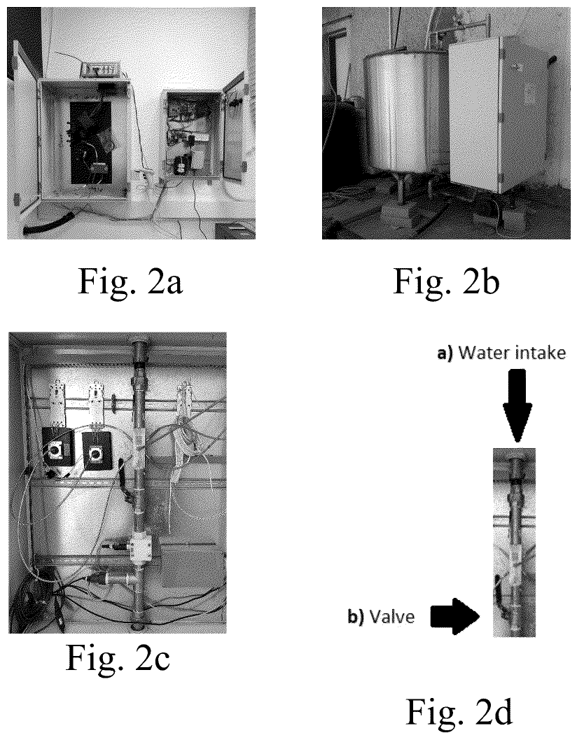

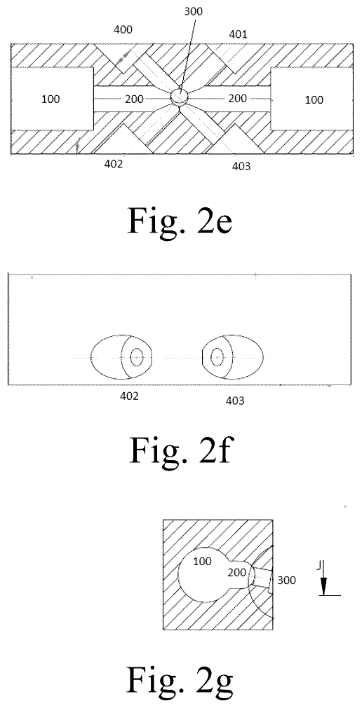

Reference is now made to FIG. 1 presenting a block diagram of a measurement setup. The water is circulated in the circulation Jig by a pump (D), Passing through an optical measuring chamber (B) and sensor array (C): flow meter (C1), conductivity meter (C2) and thermocouple (C3). At the end of the cycle the water returns to the tank, where the temperature of the water is measured by. Water flows through the optical chamber (B), where it is excited by UV light sources (A). Emitted light passes to the prototype spectro-flourometer system (F) by an optic fiber straight to fiber adapter with high pass filter switch (F3). Then, light ray is decomposed to spectrum by a grating (F2), which passes right to PMT (F1). Another output of a optical chamber (B) is dedicated for transmittance UV light. This channel connected to less sensitive spectrometer (F4). All the signals, from sensors (C) and prototype spectro-fluorometer (F) pass to personal computer (H) trough control and acquisition hub (G): non optical acquisition elements (G1), optical acquisition elements (G2) and UV controller (G3). All the collected information moves to algorithm (H) for analysis. The algorithm is out of the scope of this paper, and will be demonstrated as a threshold resulted from hypothesis testing in later sections. As it seems, proposed system contains number of independent logical and physical sub systems. The setup shown in FIG. 1 consists of indoor and outdoor modules (see FIG. 2).

The water circulation jig sub-system contains three key elements: water tank, pipe system and PKm60 pump (see FIG. 1, D) (Pedrollo, Italy). Tank has a 1000 l volume, therefore may contain sufficient amount of substance for large scale tests. It is custom made from stainless steel. The tank is connected to the circulation system through 1″ pipes system. Pump connected to a simple switch, and flow velocity controlled by a valve, and monitored by flow rate sensor that detailed explained in (2.3. Maximum supported flow rate is

$${40}^{\frac{l}{m i n}} .$$

This sub system located entirely outdoor, in order to simulate field conditions for experiments.

In one mode of operation the circulation is stopped. By closing a valve below the optical chamber one can perform low volume calibration test by purring small amount of test solution from an entrance in the top of the column (water intake) FIG. 2d. For the sake of calibration, the column may be worm up by flexible heating element wrapping around the optical chamber. Water temperature controlled by a temperature gage inserted from the top water intake.

There are tree sensors that serve for system control and environmental data collection. First sensor is DRH-1X50G6N6F300 (Kobold, Hofheim, Germany) flow meter (see FIG. 1, C1) that measures in range a

$$2.5 - 50 \frac{l}{min} ,$$

which covers possible substance flow. It used for indication of pump stability and, as we'll see later, possible substance density measurement. For temperature estimation standard PT-100 thermo-couple with M-345 (Michshur, Holon, Israel) transmitter (FIG. 1, C3). Thermo-couple is mounted in substance tank to measure overall temperature, and is not affected by flow. The last sensor is BlackLine CR-GT with corresponding microprocessor ecoTRANSLf-01(JUMO, Fulda, Germany) which measures conductivity (FIG. 1, C2) of a substance and has a built-in temperature correction. First two sensors are mounted in pipes, while later in a water tank. All sensors are connected to Arduino Mega2560 (Arduino, Turine, Italy) (FIG. 1, G1), which connected to PC (FIG. 1, G,H). General images of the outdoor circulation system, the prototype fluorimeter and control hub, presented in FIG. 1.

The core of the system is optical chamber that on the one hand allows to UV light pass to substance flow, and on the other not interfere with it. Assurance of both is complicated, because large water supply systems operate at high flow rate and wide pipe diameters, later will cause inner filter effect and signal will be faulty. Thus, our optical chamber is a pipe extension, with cuvette-like gap. During flow, random substance portion will fill cuvette-gap, and by this, measured flow sample remain properties of a flow as whole.

Reference is now made to FIGS. 2e to 2g presenting the design of the flow chamber. In the direction of flow there are the main flow passage (100) which in this embodiment fits to the diameter of a 1″ pipe. A second passage is of the width of 10 mm or generally at the width of a standard Quetta is used as an embedded Quetta (200). Around this passage are 5 fiber optics inlets/outlets (400-403), which may serve for nether illumination or measuring transmittance, all the inlets are on the same plane (section J-J) in the drawings. Upon need one may add more drills to attached more illumination sources, or add more sources to the system through an integration sphere which will be connected in one of the inlets (400-403) if required. In the specific embodiment two inlets serves for connecting two illumination sources and the two others as an outlets to the transmittance spectrometers. A fifth spectrometer outlet (300) is perpendicular to the plane of the other is used for collecting the fluorescence scattering also through a fiber optics. The outlet for fluorescence collection may be tilted in few degrees to lower a parasite scattering of the source illumination enter the fluorescence channel. In one embodiment to lower the this is 10 degrees to the perpendicular.

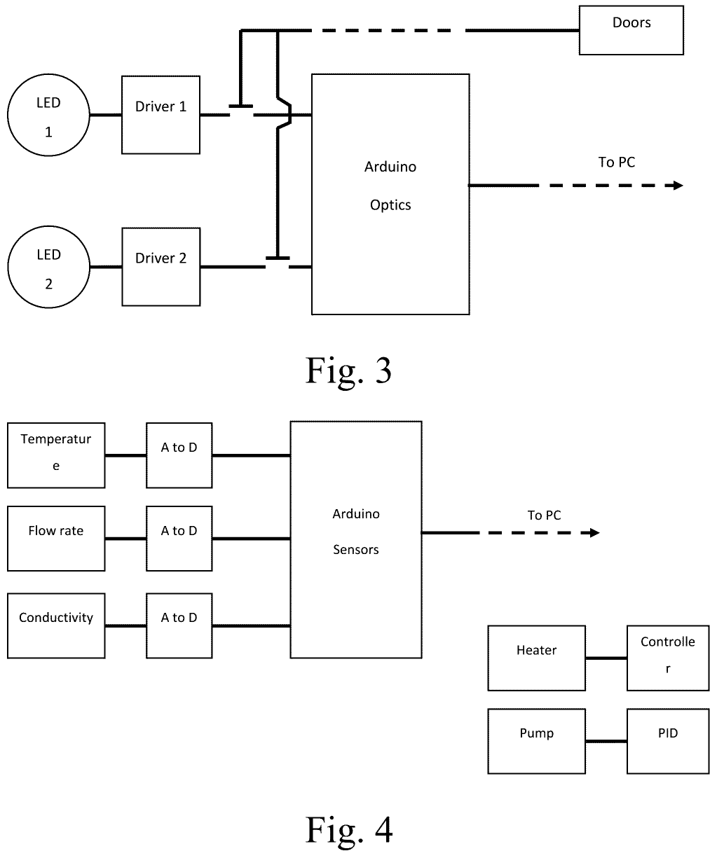

Reference is now made to FIGS. 3, 4, 5 and 6 presenting schematic diagrams of a UV source control arrangement, non-optical sensor arrangement, data processing software and a pipe system, respectively.

For water flow irradiation, optical measuring chamber (FIG. 1, C) was constructed. It is a solid aluminum block with a number of openings. There are two main openings for integration to pipe system and 5 additional SMA905 threaded sockets for fiber connection. As a result, chamber acts as a part of pipe system, without any flow interference, and allows to collect fluorescence emission and absorption in real time. Sockets 3 and 5 are connected to light sources by fiber BF20HSMA01 (Thorlabs, Newton, New Jersey, USA) with higher effective diameter, socket 7 connected to the spectrofluorometer prototype by fiber QP600-1-SR (Ocean Insight, Rostock, Germany), and an output of sockets 4 and 6 is combined to a single beam by a Y shaped fiber QBIF600-UV-VIS (Ocean Insight, Rostock, Germany) that also connected to a transmission spectrometry system.

The interaction of OM and DOM with UV radiation in 280 nm and 340 nm, resulted in typical emitted florescence spectra which is very weak and requires extremely sensitive spectrofluorometer (FIG. 1 F1). The fluorescence emission spreading in all direction, thus to minimize collection of the source UV, this measurement is done in ˜90° to the illumination source. The optical measuring chamber and the spectrofluorometer are separated. Following above, the fluorescence emission collected in the chamber is transferred to the spectrofluorometer by an optical fiber.

TABLE 1
Part list of the spectrofluorometer.
Notations are according to FIG. 3.

| | Component | | |
|---|---|---|---|
| Notation | type | Model | Manufacturer |
| A | SMA905 | SM1SMA | Thorlabs, Newton, New |
| | adapter | | Jersey, USA |
| B | Optical | SM1L05 | Ocean Insight, Rostock, |
| | tube | | Germany |
| C | Plano | LA4306- | Ocean Insight, Rostock, |
| | convex lens | VML | Germany |
| D | High pass | FGB37 | Thorlabs, Newton, New |
| | filter | | Jersey, USA |
| E | Mirror | CM254075- | Thorlabs, Newton, New |
| | | F01 | Jersey, USA |
| F | Diffraction | 41-037 | Edmund optics, Barrington, |
| | grating | | New Jersey |
| G | Mirror | 49-607 | Edmund optics, Barrington, |
| | | | New Jersey |
| H | PMT | H7260-03 | Hamamatsu Photonics, |
| | | | Hamamatsu, Japan |

TABLE 2
UV chamber sockets listing

| Socket | | |
|---|---|---|
| Number | Thread | Role |
| 1 | 1″ BSP | Flow input |
| 2 | 1″ BSP | Flow output |
| 3 | SMA905 | UV 280 nm source connector |
| 4 | SMA905 | UV 280 nm Absorption collector |
| 5 | SMA905 | UV 340 nm source connector |
| 6 | SMA905 | UV 340 nm Absorption collector |
| 7 | SMA905 | Emission collector |

For the sake of measuring the weak fluorescence emotion, emitted out of the optical chamber, a spectrofluorometer prototype was built. The spectrometer is a modification of Czerny-Turner arrangement. The optical design preformed in few steps: first, based on assumption on the expected concentration, a course evaluation of power loss and signal to noise ratio was done. Having that we simulate the spectrometer behavior using OSLO Pro (Lambda, Littleton, Massachusetts). The expected diffraction behavior of the spectrometer, as well as, the initial values for focal lengths estimated by MATLAB simulation of the diffraction grating (Mathworks, Natick, Massachusetts). The system built in steps, where each subcomponent was checked separately.

Reference is now made to FIG. 3 presenting an optical arrangement of the spectrofluorometer. The spectrometer feed through a fiber optic cable connected to SMA905 adapter (A), the 22° light of cone was coupled to a plano-convex UV lens (C) through an optical tube (B). To reduce parasite emission associated with the source UV, the outcome wave-front passes through a two stops filters wheel (D) that controlled by Arduino Uno R3 (Arduino, Turine, Italy), with two high pass filters from a same type. The filters crossed to optimize truncating the parasite light, and flipped according to the wavelength of the light source. Next the filtered wave-front hit a first mirror (E), resulted in a collimated light propagate to a diffraction grating (F) with 1200 lp/mm. Finally, by the aid of a second mirror (G), the dispersion pattern was focused on a PMT (H) 32 channel line detector allowing of instant monitoring of entire emitted fluorescence spectrum while the water is in flow. The data acquisition of the PMT's readings done by IQSP480 acquisition system (Vertilon Corporation, Westford, Massachusetts, US) (FIG. 1, F1) which connected to PC (FIG. 1, F and G). The principal components of the spectrofluorometer system listed in Table1.

Natural florescence of tryptophan and humic acids are characterized by a low quantum efficiency, typically of the order of few percentage. Thus, the optical signals are weak, the spectrometer losses are invariant to the power of the fluorescence. However, if the power is too low the PMT would not be able to identify the signal. Given and PMT with potential gain of 2.106, the burden of providing sufficient signal fall on the efficiency of illumination and light collection in the optical chamber.

Excitation of tryptophan is 280 nm with emission at 335-365 nm, while for humic origins best excitation wave is 340 nm with emission at 400-480 nm. Since fluorescence power is within proportion to concentration of the active material, the determination of required power relayed on testing of real substance. These experimental results suggested that the required optical power has to be at least 25 mW. To meat that we chose light sources from deep ultraviolet LEDs array series DUVXXX-SD356LN (Roithner Lasertechnik, Vienna, Austria) with nominal optical power (Po) 26 mW and 32 mW respectively. The sources bandwidths (FWHM) are 10±2 nm. LEDs were integrated to printed circuit boards (PCB) SD35-PCB (Roithner Lasertechnik, Vienna, Austria), that mounted in aluminum housing which is consists of mounting plane and cover.

The DUVXXX-SD356LN assembly includes concentration lens. Following the manufacturer this resulted in concentration of 50% of the radiation power in a field of view (FOV) of 40°. As a result, coupling of sources to standard 22° FOV 600 μm core fiber optics was poor. To cover most of the radiation the single core fiber was replaced by a 2 mm diameter bundle of seven 550 μm cores, which was found sufficient experimentally. Furthermore, to reduce parasite radiation in unrequired bandwidth, a bandpass filter was added to the illumination sources. 35-881 (Edmund optics, Barrington, New Jersey) for 280 nm LEDs array, and 65-129 (Edmund optics, Barrington, New Jersey) for 340 nm LEDs array. For the sake of measuring the emitted spectra with the two sources, the LEDs array were alternate in 1/15 Hz.

Coupling of the LEDs array to the fiber optic required 3D adjustment. Using SM1SMA (Thorlabs, Newton, New Jersey, USA), the fiber optic cable was mounted to the LED through SMA905. The distance between the fiber optic and the emitted surface done using the SM1 thread. Parallel adjustment done using while tightening the treads. To maximize the coupling the adjustment done while measuring the illumination with a spectrometer. To insure LEDs working temperature, ‘each light module supported by a heat sink (ABL, Wednesbury, UK).

We have two optical fiber cables that enter two spectrometers. One optical fiber carries absorption optical signal and goes straight to Flame-T-RX1 (Ocean Insight, Rostock, Germany) (FIG. 1 F4) spectrometer that connected via USB cable to PC (FIG. 1, G). It is used for system calibration and operation of it is not discussed further in this paper.

As mentioned above the PC (FIG. 1, G and H) connected to fluorescence acquisition system (FIG. 1 F1), controllers (FIG. 1, G1 and G2) and absorption spectrometer (FIG. 1, F4). Both controllers are Arduino boards that are programmable, other products have either software APIs or separate software. The IQSP480 comes with software that allows to control gain, sample frequency and log data. Data written in CSV format with a time stamp. The FLAME has Python API, which allows to access device parameters and acquired spectrum data. The Arduino boards were programmed according to pre defined tasks and log every step. The data resulted in tree logs files: UV states, fluorescence signal and sensory data. At the end of each experiment only two logs file were processed, so that active fluorescence and sensory data are synchronized. UV state log serves for calibrations only.

The signal S(t) resulted from the Fluorescence emission was written to the PC continuously and post processed Post processing begins with normalization of an signal by subtraction of clean water fluorescence signal Eq. 1. It expected to be zero for every wavelength, therefore it is suits the task of removing unwanted artifacts. Due to measurement range, excitation source of 340 nm produces scattering that excides maximum value even of the emission produced by 280 nm source. By exploiting this information, one may rely on simply hypothesis test—calculation of maximum values for each normalized excitation wave and mean value. Mean will be used as a threshold, or separation hyperplane, that spread fluorescence signal produced by each wavelength. To reduce noise in resulted sets of signals running mean is calculated on every 1500 spectral signals, without overlap. 1500 signal are collected in a course of 30 seconds, which in a typical flow rate of

$$40 ( \frac{L}{min} )$$

resulted in a measurement for every 15 L passes through the system.

Reference is now made to FIG. 4 presenting a flowchart of the post processing workflow. Where two pairs signal types are acquired: S_{280,m},S_{340,m} are mean values of spectrum per excitation and time and M₂₈₀, M₃₄₀ are maximum values of S_{280,m}, S_{340,m} respectively.

$$\overline{S ( t )} = S ( t ) - {S}_{w} \qquad ( 1 )$$

Just as fluorescence signal, the other sensory data had noise resulted from the harsh measurement conditions. To overcome this issue sensory data is smoothed by second order of Savitzky-Golay filter.

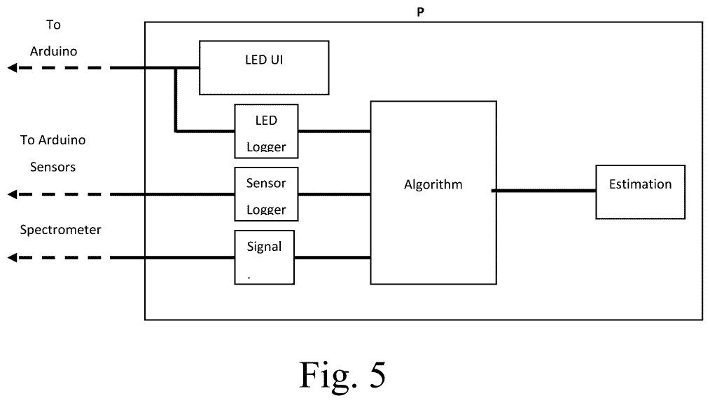

Prior to the assemble, UV sources, fiber cables, and spectrometer system were tested on temporal workbench. Test required for light source and in one point required for a natural florescence source which could mimic the real-world DOM break. Searching for available, and safe material we have found the diluted 3% fat cow's milk (“Tnuva”, Herzeliya, Israel) suitable for the task. Apart of its availability, the milk was chosen due to its high concentration of proteins, that rich with amino acids, especially tryptophan. I.e., it simulates expected TLF peak where DOM is active. Milk was injected to cuvette in ration of 10⁻⁴ milk to water. Process of signal acquisition of such a substance is a very easy task for standard laboratory fluorimeter, therefore it suit as a baseline for our system setting. The expected tryptophan concentration in our dilution is 40 ppb. Reference si now made to FIG. 5 presenting milk mixed with Double distilled water (DDW) fluorescence signal excited by different UV sources. While with excitation of 280 nm TLF peak is clearly visible, 340 nm reveals little or no change at all. In addition to described experiment, milk will be also used in full scale experiments.

The system includes three non-optical sensors, where each of them had to be calibrated. Firstly, flow meter calibration was performed in a course of 12 experiments with different flow rates that were manually changed by adjusting pump voltage. Temperature and conductivity meters were calibrated in a same experiment that was performed during nine days. The first four days were dedicated to stability test of a system, and last five days were aimed to test and calibration of conductivity meter by daily adding of sodium chloride (NaCl) to the water tank. To determine the required salinity range. Using a calibrated portable field conductivity meter CD-4303 (Lutron Electronic Enterprise, Taipei, Taiwan), the level of expected salinity range was predetermined by measuring samples of irrigated water source from various sources. The same instrument used as a reference to sample the salinity through the experiment. Compering the measurements of the inline sensor to the portable sensor used for calibrate the inline sensor, this resulted in a correction function Eq.2 which realized as a post processing correction on Arduino Mega2560. Where each parameter was estimated empirically from measured errors and Ĉₘ is after compensation and Cₘ before.

$$\hat{{C}_{m}} = {C}_{m} \cdot 1.0244 + 0.0572 \qquad ( 2 )$$

For the sake of calibrating the in-line spectrofluorometer, the measurements of the proposed system compered to measurements taken by a lab graded calibrated spectrometer FLAMET-RX1 (The calibration in the required bandwidth (300-520 nm) relay on measuring SL1-LED (StellarNet Inc., Tampa, Florida, USA) LED kit. Measuring the kit with the two spectrometers. One should know that when scale of nm/bin is scaled, two calibrated spectroscopes should resulted with the same spaces when measuring a series of the same calibration LEDs, showing the same profile to each of the calibration LEDs.

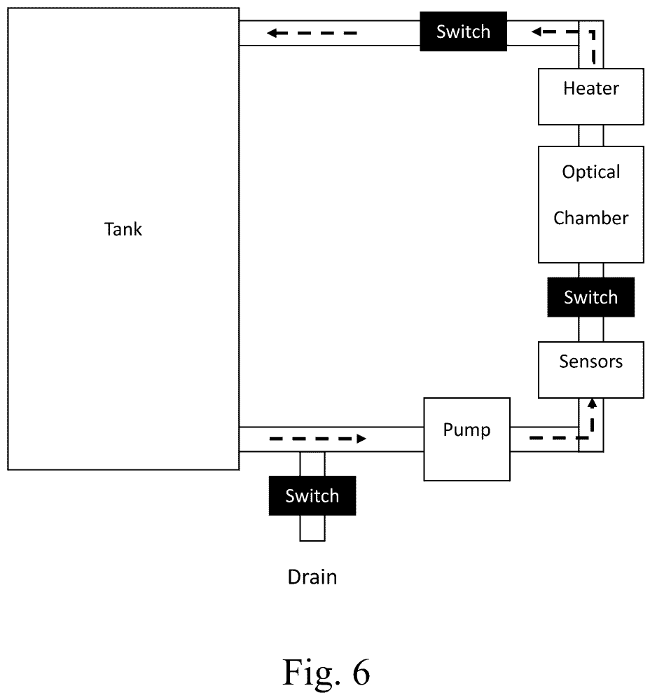

Reference is now made to FIG. 6a presenting native measurements taken by the two systems. Observing the figure the in-line spectrofluorometer and the reference spectrometer forms a different signal shape, with a relative shift which is wavelength dependent. Therefore, calibration routine should be performed to overcome wavelength error.

Two issues arise at such a procedure: (1) FLAME and PMT have different resolution (2) Light sources have different intensities. For former issue solution is relative simple—decimation of a signal to 32 bins. Later is solved by separate normalization of each signal by Eq. 3.

$${S}_{norm} = \frac{S}{max S} \qquad ( 3 )$$

To evaluate the wavelength dependent shift, cross-correlation was calculated for every PMT-FLAME signal pair (S_{FLAME,i},S_{PMT,i}). The shift δ(λ) determined according to the maximum of cross-correlation operation. A wavelength dependent formula for the shift derived by preforming polynomial approximation fit to the shift value as function of wavelength or channel (λᵢ) Eq. 4 parameters are estimated and it is implemented for signal correction.

$${λ}_{drift , i} ( {λ}_{i} ) = {a}_{0} + {a}_{1} {λ}_{i} + {a}_{2} {λ}_{i}^{2} + {a}_{3} {λ}_{i}^{3} \qquad ( 4 )$$

Yet, λ_{drift}(λ) is not a wavelength axis correction, but a step correction as shown in Eq. (5) where Δλ is contains step of decimated signal(Ŝ_{FLAME,i}) and correction of full pipeline is presented in a form of algorithm (1), which implemented in SciPy.

$${λ}_{PMT , i} ( {λ}_{drift , i} ( {λ}_{i} ) , Δλ ) = {λ}_{FLAME , i - 1} + Δλ \cdot {λ}_{drift , i} ( {λ}_{i} ) \qquad ( 5 )$$

Algorithm 1: Wavelength correction for optical signal,

```
Result: Corrected wavelengths \hat{λ}(λ)
Initialization: N corresponding series of signals S_{PMT,i} and
  S_{FLAME,i}; for i = 1 to N do
  | Ŝ_{FLAME,i} ← Decimate S_{FLAME,i} ;
  | Ŝ_{PMT,i}, Ŝ_{FLAME,i} ← Normalize Ŝ_{PMT,i}, Ŝ_{FLAME,i} by Eq. 3 ;
  | Δλᵢ ← max(Ŝ_{PMT,i} ★ Ŝ_{FLAME,i}) (τ) ;
end
Approximate \hat{λ}(λ) from series of Δλᵢ by Eq. (4) ;
Estimate λ_{PMT,i} by Eq. (5) ;
```

Reference is now made to FIG. 6b presenting data corrected on the basis of Eq. 5. Observing the figure the correction is significantly reduces the wavelength dependent shift, where the centers of the measurements on each wavelength are closely matched. The average shift error reduces from 14.4 nm before the correction to 2.4 nm after the correction which is ˜34% of the spectral resolution of the spectroflourometer prototype, which is expectable error.

For the sake of signal amplification, the PMT requires for a reverse voltage bias. Effective signal form was acquired with gain of −900 volt and integration time of 500 μsec with sampling rate of 79 Hz.

For the sake of testing the proposed spectro-fluorometer. Samples of mixed irrigation water taken from various irrigation reservoir was measured both by the proposed system and in standard lab graded spectro-fluorometer RF-5301PC (Simadzu, Kyoto, Japan) A comparison of the measured fluorescence spectra of irrigated water with 280 nm excitation presented in FIGS. 8a and 8b.

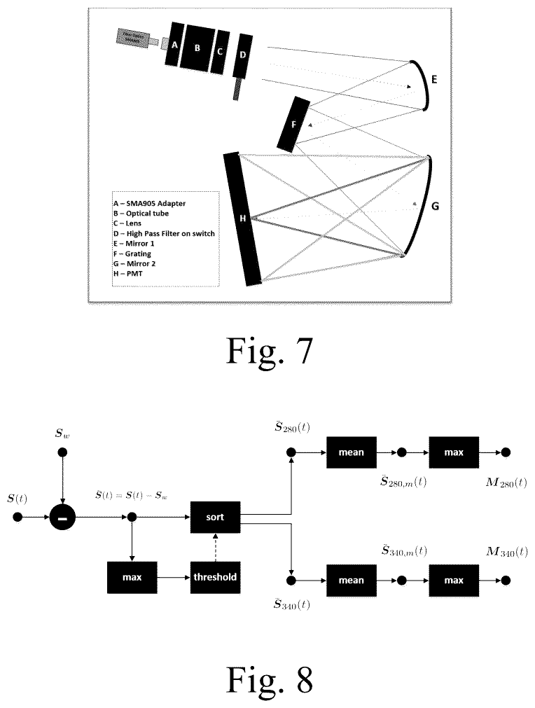

Reference is now made to FIG. 7 presenting the experimental results measured by the prototype which resemble the measurements done in the lab. There was some deviation in measurement which may be related to the fundamental difference in measuring conditions in lab system and in outdoor conditions which depends on differences in the environmental conditions, calibration conditions, and cleaning condition of the outdoor system. The outdoor system subjected to accumulation of biofilm residue and sediments which does not exist in lab cuvette. FIG. 7 exemplifies fluorescence signal of treated water for irrigation measured by laboratory flowmeter and by proposed system. Observing the figure, the prototype follows the lab measurements, showing an accurate response in the region of the TLF peak (˜335 nm), and inaccurate response in the region of the HLF peak (˜440 nm). Bear in mind that DOM dominate by biological loads, we assumed that the accuracy in the TLF is the most important and that HLF response is less significant.

The built system was tested in tree scenarios. In each case milk was injected to base substance as simulation of dissolved organic matter contamination. In the first case the base substance was clean tap water under excitation wave of 280 nm, in the second the base substance was threaded irrigated water under same excitation wavelength. The third scenario summarizes system evaluation by implementing both UV sources i.e., excitation in 280 nm and 340 nm in alternating manner, with irrigated water as a substance and milk as a DOM contamination.

Initial experiment based on various concentrations of milk and only one UV source of 280 nm is used. Milk was added to a 200 l of tap water every 24 hours for overall 72 hours of experiment, simulating a break of DOM into drinking water system. Plan of milk injection presented in Table 3. Sensory data was recorded constantly with sample frequency of 5 minutes, while fluorescence was recorded for 30 min with intervals of couple of hours within day hours. Expected fluorescence signal form successfully measured, compared to laboratory results (FIG. 5) witch same excitation wavelength.

TABLE 3
Milk experiment plan

| | Day | | |
|---|---|---|---|
| | 1 | 2 | 3 |
| Milk to water ratio | 0.0 | 1 · 10⁻⁴ | 2 · 10⁻⁴ |
| Milk injection volume [l] | 0.0 | 0.02 | 0.02 |
| Expected tryptophan ratio [ppb] | 0.0 | 40 | 80 |

Due to high environmental temperature (above 30° C.) one may assume that bacteria reproduction rate increased in a second day of experiment, which led to un-predicted fluorescence behavior—maximum values started to climb (FIG. 8) by temperature value opposing expected reduction. Yet, this phenomenon allowed estimation of time constant for fluorescence signal dynamics and validate proposed effective source alternation rate. Every transition state lasted for at least of 2 hours, with minimal time constant of 40 minutes. In addition, by conducted tests effect of temperature on LEDs is neglected.

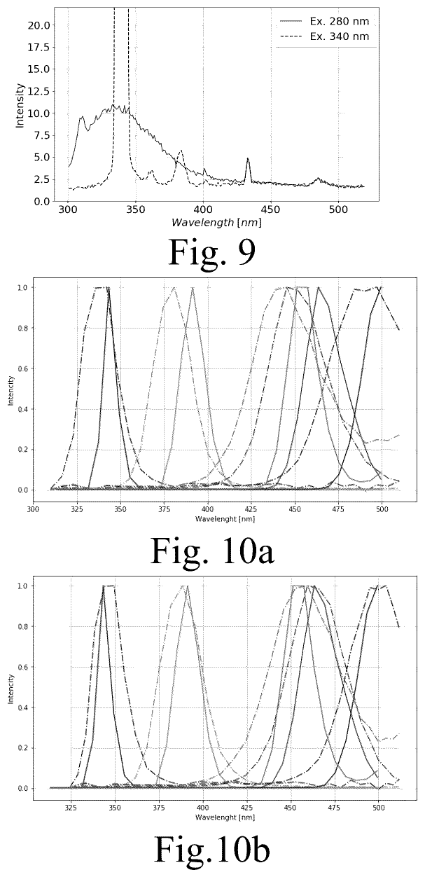

Reference is now made to FIG. 9 presenting typical fluorescence response of the tap water (in dashed dashed), and the tap water after DOM breach of 40 and 80 ppb concentration (blue and dashed red, respectively). Since response is not temporally fixed one can characterized each signal by its distribution. Assuming a Gaussian distribution one can determine an optimal threshold using a hypothesis testing.

Second experiment was performed in a similar manner to previous one, but in this case treated irrigation water from functioning water reservoir of TsabarKama co-operative (Revadim, Israel) was used as the base substance and both excitation wavelengths were activated. The optical data and fluorescence signal were recorder 12 hours per day and reference samples, for florescence and microbial count, were taken four times a day: morning, twice at afternoon (before and after milk injection) and at evening.

Reference is now made to FIG. 10 presenting experimental results of milk fluorescence. It should be appreciated that milk fluorescence is easily distinguishable from a base substance visually, and as proposed above.

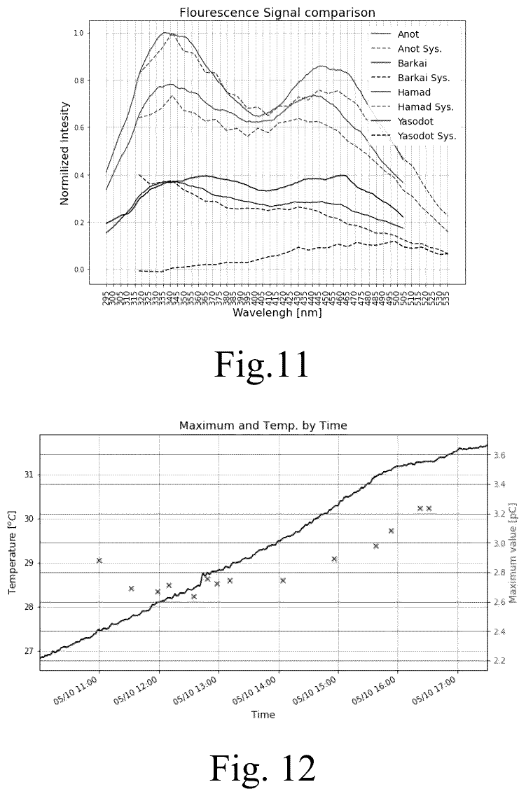

Reference is now made to FIG. 11 presenting maximal TLF response in time which is not typical for fluorescence emission, because peak value expected to diminish with a rise of temperature. By observing the FIG. 12, and by our assumptions, this is effect of changing biological dynamics within the substance that are reflected in the acquired signal.

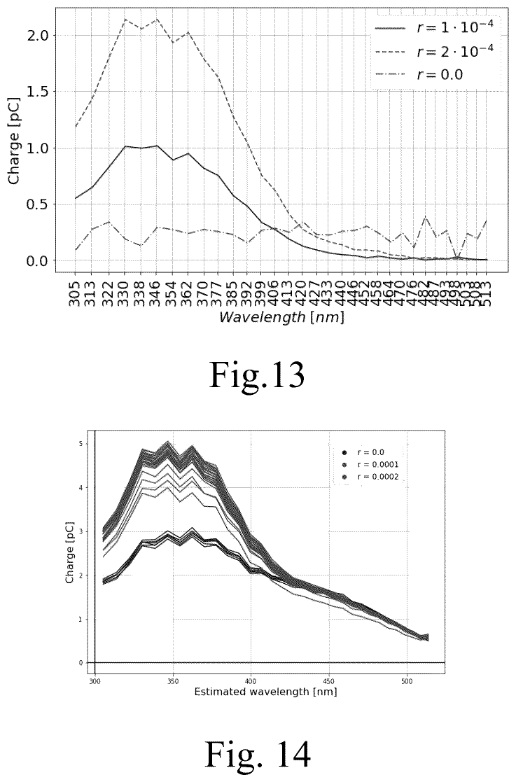

Reference is now made to FIG. 12 presenting experimental results for 280 nm and 340 nm excitation. FIG. 13 presents fluorescence spectrum hour before injection, hour after and 24 hours after injection. As expected, TLF peak risen after milk injections whereas HLF has not changed as expected. Moreover, strange behavior that was observed previously returned. The system successfully recorded the signals form as in previous experiments. The maximum fluorescence values as function of time, to the two excitation wavelengths presented in FIG. 14. The results resemble the previous finding, the system is sensitive to DOM breach. As in section 4.2 we see a dynamical change i=n maximal value as function of time.

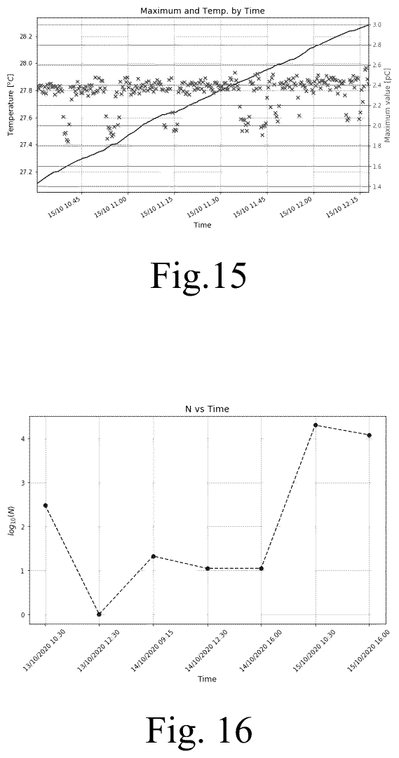

During our experiments non optical data was collected. Despite some minor change in flow speed, which, by our assumptions are result of temperature change, numbers are stable, and no anomalies were found. Example of such a data from experiment is in FIG. 15. Sensory values are smoothed for a course of 48 hours. The vertical green dashed line indicates milk injections time. FIG. 15a presents flow speed measurements, 15b temperature measurements of the waters in the tank and 15c water conductivity.

One must be careful with a left overs of substance on equipment. Despite cleaning with high concentration of Ca (ClO₂), biofilm was found on fiber and resulted in fluorescence signal. It was reduced after additional cleaning with fine wipes, but still remained (see FIG. 16).

System built upon on two excitation waves (280 nm, 340 nm), emitted from UV LED sources, custom made optical measuring chamber, that suit for various pipe diameters and shapes, common optical system configuration and a PMT. It provides 32 channel fluorescence spectrum signal that shows TLF and HLF with complimentary sensory data—substance temperature, conductivity and flow rate. Prototype was able to successfully measure fluorescence of irrigated water, and detect simulated OM breach by milk injections equivalent top 40 ppm of tryptophan by analysis of TLF. Furthermore, due to constant recording unexpected fluorescence spectrum behavior was revealed, thus system also grants monitoring of solution's biological and chemical dynamics that are reflected in fluorescence spectrum.

Given a physical world model xᵢ=h_{w}(Pᵢ) where xᵢ∈R^{d} is single observation (Ex. spectroscopy measurement) and Pᵢ∈ [symbol] ^{S} is the available world state data of environmental variables (Ex. Chemical concentration) with rank S. The world state may be separated into two sets of parameters yi and pi, where yᵢ∈ [symbol] ^{S-K} composed of S-K response values to be estimated, and pᵢ∈ [symbol] ^{K} composed of K environmental parameters accounted in the physical model which describe the relation between the observations-response, denoted ƒ_{w}. In this formulation, the estimation task is to find function ƒ_{θ} with a set of tunable weights θ that minimizes pre-defined loss function L such that:

$${y}_{i} = {f}_{w} ( {x}_{i} , {p}_{i} ) \\ {\hat{y}}_{i} = {f}_{θ} ( {x}_{i} , {p}_{i} ) \\ {θ}^{*} = {argmin}_{θ} [ ℒ ( {y}_{i} , {\hat{y}}_{i} ) ] \qquad ( 1 )$$

First obstacle on the way of estimation is measurement noise σₑₙᵥ that effects our knowledge of response variable:

$${y}_{i} = {f}_{w} ( {x}_{i} , {p}_{i} ) + {σ}_{env} , {σ}_{env} \sim {p}_{σ} \qquad ( 2 )$$

The estimation process is subjected to few main challenges. The estimator to ƒ_{w} is ƒ_{θ} since ƒ_{w} is mostly non-linear, the estimation may be non linear with reference to ƒ_{w} or linear after applying non-linear transformation to (xᵢ,Pᵢ). In both cases, the form of the physical model ƒ_{w} has to be known, analytically or empirically. Additional complications originate from the measurement equipment, while we are interesting in the observations true values xᵢ, due to the limited measuring accuracy the measured values x̅ᵢ include some errors relative to xᵢ. In addition, measurement equipment F_{HQ} is affected by the environment resulted in changing of parameters (Pᵢ), through the measurements, the overall measurement structure is:

$${\overline{y}}_{i} = {f}_{w} ( {\overline{x}}_{i} , {p}_{i} ) + {σ}_{env} ( {p}_{i} ) , {\overline{x}}_{i} = {𝔽}_{HQ} ( {x}_{i} , {P}_{i} ) \\ {\hat{y}}_{i} = {f}_{θ} ( {\overline{x}}_{i} , {p}_{i} ) \\ {θ}^{*} = {argmin}_{θ} [ ℒ ( {y}_{i} , {\hat{y}}_{i} ) ] \qquad ( 3 )$$

Upon changes in environmental conditions the true world physical model ƒ_{w} resulted in different measuring values to the same inputs. Measurements in aquatic environment may be influence by a physical parameters such as turbidity, temperature, and solvent PH, while this values kept reasonably stable in lab conditions Eq. (2). it may subjected to a significant variation under outdoor environmental conditions Eq. (3), which may affect the measuring values. In addition, as shown in Eq. (3) above, changes in measuring condition may resulted from a change of the measuring instrument.

A common case of influence of equipment on the measurements, is utilization of a outdoor measurement equipment F_{LQ} which characterized by higher measurement error, and lower scan resolution that results in low quality samples

$${\overline{x}}_{i} ∈ {ℝ}^{{d}_{LQ}} , {d}_{LQ} < {d}_{HQ} .$$

Where d_{LQ} denoted the number sampling points in a low-quality measurement, and d_{HQ} is the number of sapling points in a high-quality measurement.

Since measurements Eq. (3) influence by many factors estimating of the physical process is highly complicated scenario.

One of the most important challenges in water quality monitoring, is estimating of organic matter (OM) concentration, and dissolved organic matter (DOM). This is estimated by the analysis of tryptophan-like-fluorescence (TLF) at 280/350 nm and humic-like-fluorescence (HLF) at 320-360/400-480 nm emission/excitation pairs may reveille the level of contamination. In addition, temperature has an effect on solution, and through this on a signal, such that data collection for model training has to consider both concentration (c) and temperature (T).

There are a variety of high-grade fluorimeters that typically have high signal resolution

$$( \text{e.g. Resolution} \leq 1 \frac{nm}{bin} )$$

and high SNR values. Meanwhile, in field conditions photomultiplier tubes (PMT) are used due to low signal energy. They perform well in signal detection, yet there are limitations on resolution. Data collection is based on specific setting described below.

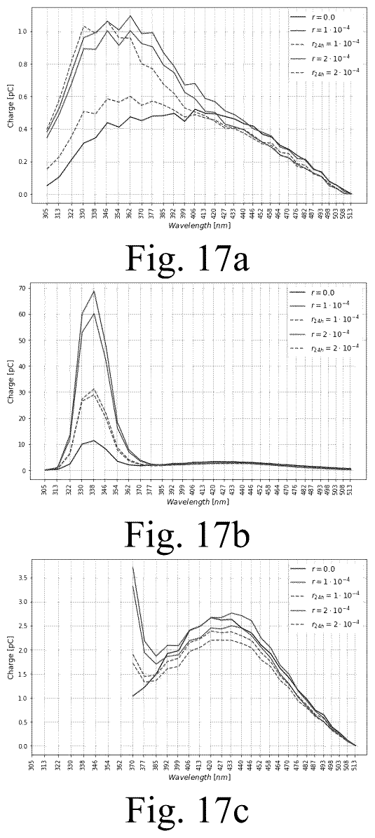

Data was collected for system training and evaluation, summed up to 1190 EFS of tryptophan in double distilled water (DDW) samples. Sampling was performed on laboratory equipment RF-5301PC (Shimadzu, Kyoto, Japan) with high resolution capabilities (Resolution=1 nm/bin). To allow real evaluation of the method's robustness, training set and test sets were taken in different c-T sets (Table 1), ten samples for each couple, with a test-train ratio of 36%-64% (800 train samples and 290 test samples). Thus, data is balanced. EFS wavelength range was limited to [280,500] nm, i.e. (d_{HQ}=221). Samples in various c-T couples presented in FIG. 17, observing the figure different c-T couples appear almost identical EFS, thus, ƒ_{w} is not linear.

TABLE 4
Concentration and Temperatures that
were used for data sets sampling.

| | |
|---|---|
| Train values range | Test values range |

| c[ppb] | T[° C.] | c[ppb] | T[° C.] |
|---|---|---|---|
| 500.62 | 22.0 | 599.80 | 20.5 |
| 475.05 | 27.0 | 550.75 | 25.0 |
| 449.84 | 29.0 | 490.14 | 30.0 |
| 400.05 | 35.5 | 426.53 | |
| 350.01 | 40.0 | 325.32 | |
| 300.16 | | 175.24 | |
| 249.62 | | 149.27 | |
| 199.27 | | 89.80 | |
| 149.11 | | 29.98 | |
| 99.61 | | 8.18 | |
| 74.79 | | 3.18 | |
| 50.37 | | | |
| 24.98 | | | |
| 14.99 | | | |
| 5.00 | | | |
| 2.27 | | | |

Left—Train set,
Right—Test set

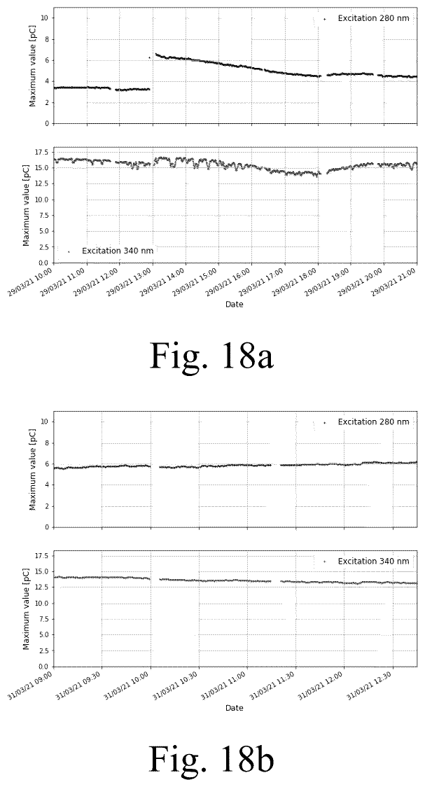

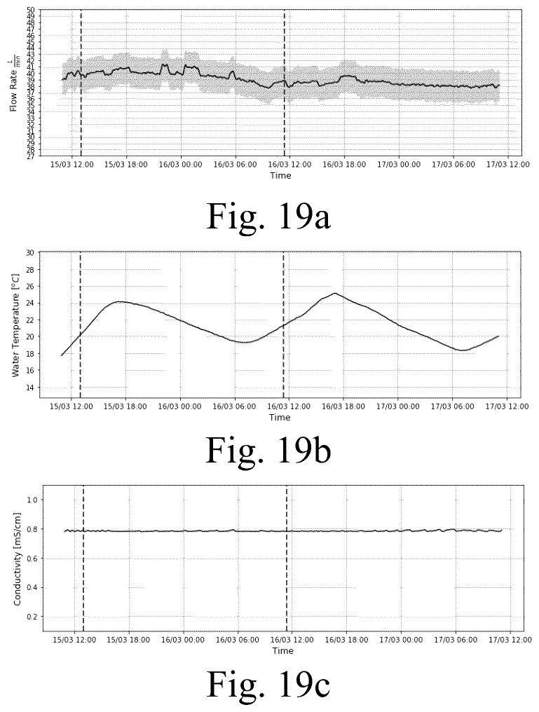

To simulate real world environment, one must take into account equipment limitations, and the physical structure of a sample. The motivation for simulation is based on our project where we are building an outdoor 32 channel spectrofluorometer aim to measure in flow the quality of irrigation water. The data equitation system of that prototype relies on the IQSP480 acquisition system (Vertilon Corporation, Westford, Massachusetts, US) and the photo multiplayer array H7260-03 PMT (Hamamatsu Photonics, Hamamatsu, Japan). To mimic the expected data, number of synthetic data sets types were created. All of them are in low resolution, namely they were matched to output dimension of 32 channel, thus the high-quality data (d_{HQ}=221) reduced to 32 (d_{LQ}=32) by nearest neighbor technique, Next, another data set was built upon low resolution samples. This time, signal shape is modified to match fluorescence of real-life solution (irrigation water), an example to high quality measurements of irrigation water shown in FIG. 18, which composed of double peak structure. The true concentration values in a real-life solution is not known, thus we adapt concentration of a laboratory acquired signal with a similar fluorescence peak response. The modified samples are made to simulate this behavior—TLF is shifted to 335 nm and HLF peak (second peak) generated by Gaussian-like function with mean drawn from uniform distribution λ_{HLF}˜U[440,450] just as amplitude A˜U as described in Eq. 4. Comparison of original signal and a double peak sensitized version (augmented) is observed in FIG. 19. Estimated SNR of low-quality data is relatively high SNR_{db}≈5 db, therefore signal was not modified in means of noise.

$${HLF}_{peak} ( x ) = A \cdot {e}^{- {( x - {λ}_{HLF} )}^{2} / 2 {σ}^{2}} \qquad ( 4 )$$

Following above, our attention is using network suitable to handle both high quality and low-quality data sets. Assuming that the high-quality and low-quality data sets share the same features set, the training of a model has two steps: single domain training, with high-quality data, and transfer learning step. In single domain training mode, an encoder (E) is trained to tie samples to some set of features with a lower dimension which can support a transformation both from high quality data and from low quality data. Next this set of latent features {Zi} is mapped to concentration estimation {y̅i} through a regression operator (R). To support low quality data, at the second stage the low-quality samples tied to the same latent features {Zi} through a new encoder while the regression operator does not change. The overall network structure denoted (ER). In our scheme, the encoded latent space of {Zi} is in a lower dimension relative to both high quality data and low-quality data, to inforce the spread of the latent space model training done while minimizing a contrastive loss function, which suits the most.

Siamese networks combined with contrastive loss proposed for dimension reduction, where sample dimension is d_{HQ} and latent is d_{z} where (d_{HQ}>>d_{z}), preserving latent manifold. Let Dᵢⱼ be the distance between two latent variables zᵢ and zⱼ, which are output of Siamese encoder E_{φ} such that:

$${z}_{i} = {E}_{φ} ( {x}_{i} ) , {D}_{ij} = {‖ {z}_{i} - {z}_{j} ‖}_{2} , {d}_{z} < {d}_{HQ} \qquad ( 5 a )$$

Where φ denoted the encoder parameters. The contrastive loss L_{cont} will have a following expression:

$${ℒ}_{cont} = \frac{1}{2} [ ( 1 - {s}_{ij} ) \cdot {D}_{ij}^{2} + {s}_{ij} \cdot max {\{ 0 , m - {D}_{ij} \}}^{2} ] \qquad ( 5 b )$$

And sᵢⱼ denoted as the similarity factor between two (xᵢ,xⱼ) samples

$${s}_{i j} = \{ \begin{matrix}1 , {c}_{i} \neq {c}_{j} \\ 0 , {c}_{i} = {c}_{j}\end{matrix} \qquad ( 6 )$$

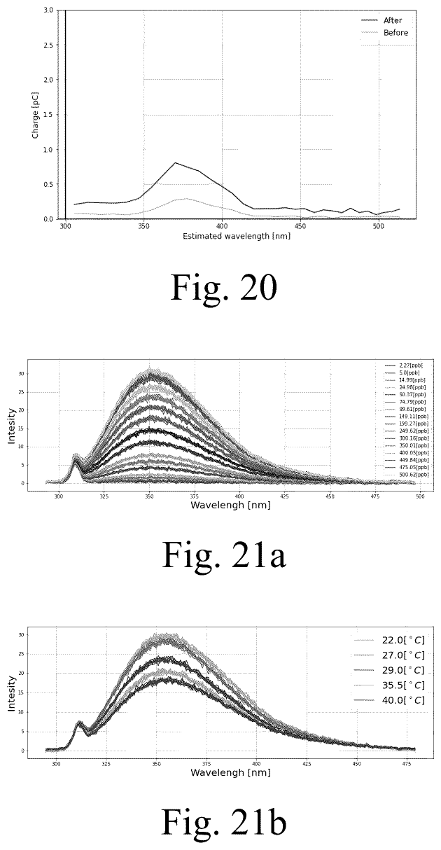

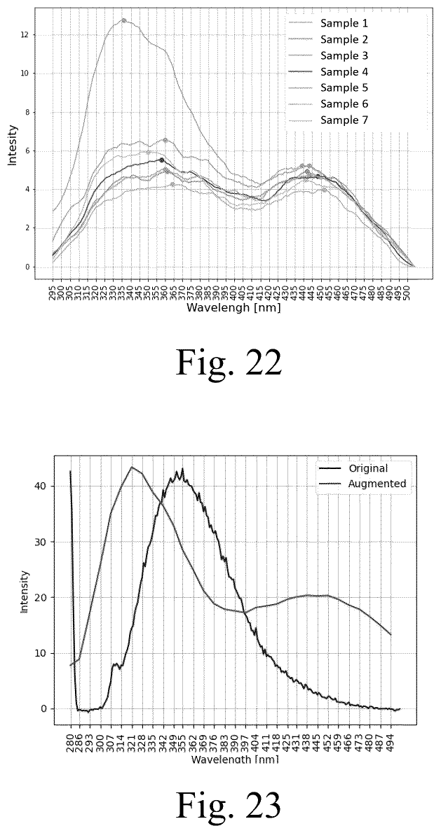

L_{cont} minimizes distance (Dᵢⱼ), when samples xᵢ, xⱼ belong to same class cᵢ=cⱼ, forces distance to match pre-defined minimal margin m when samples belong to different classes. Eventually, training is finalized when encoded samples are clustered by classes (FIG. 20). For theoretical analysis please refer to and for probabilistic interpretation of contrastive loss see appendix A. Originally the contrastive loss was suggested to tackle a classification problem, showing for dimension reduction of measured data composed of measurements originate from multiple classes. In such a setting the aim of the algorithm was to separate the low dimensions latent variables to clusters associated with their original classes. This separation achieved without any assumption on relations between the classes.

Here we suggest to look at regression as classification problem, where yᵢ=cᵢ∈ [symbol] , thus K=S=1, this implies that there are infinite classes. Since, relation between values of cᵢ connected to relation between xi, cᵢ's must be reflected on to the latent space. To inject this information, we suggest to modify only margin m by tying it to (cᵢ,cⱼ):

$${m}_{ij} = h ( \vert {c}_{i} - {c}_{j} \vert ) \qquad ( 7 )$$

Where h is an arbitrary task dependent function that defines relation between pairs of response values.

Method encodes samples to latent representation by an encoder function E_{φ} and with a help of regression module R_{ϕ} outputs desired response value. Regression and an estimator have a form of Eq. 2. Such that:

$${\hat{c}}_{i} = {f}_{θ} ( {x}_{i} , {p}_{i} ) + {σ}_{e n v} \qquad ( 7 b )$$

In addition, we use all available prior knowledge about measurement state in a form of vector Pᵢ∈R^{S}. The dimension of latent variable is defined by the number of known environmental values zᵢ∈R^{S}. It is possible to rewrite zᵢ as:

$$zi = [ zi , 1 ( {x}_{i}^{-} , {P}_{i , 1} ) , … , zi , S ( {x}_{i}^{-} , Pi , S ) \qquad ( 8 )$$

Note that response value is also belongs environmental variables yᵢ⊆Pᵢ.

Substitute Eq. (7) in Eq. (5b) modified contrastive loss will be applied independently for every environmental variable and summed as mean of losses:

$${ℒ}_{cont , s} = \frac{1}{2} [ ( 1 - {s}_{ij} ) \cdot {D}_{ij}^{2} + {s}_{ij} \cdot max {\{ 0 , h ( \vert {c}_{i} - {c}_{j} \vert ) - {D}_{ij} \}}^{2} ] {ℒ}_{E} = \frac{1}{S} {\sum }_{s = 1}^{S} {ℒ}_{cont , s} \qquad ( 9 )$$

To estimate the concentration value c̅, theregression loss is applied in a form of mean absolute error (MAE). The MAE was chosen due to its robustness to outliers:

$${ℒ}_{R} = \frac{1}{N} {\sum }_{i = 1}^{N} [ \vert {\overline{c}}_{i} - {\hat{c}}_{i} \vert \qquad ( 16 )$$

To realize constrictive loss (Eq.5b) one is training siamese encoders, the outcome of the encoders are two latent variables zᵢ, zⱼ. Each latent variable is passing through the regressor R_{φ}, each of which resulted in an estimation for the concentration values ĉᵢ, ĉⱼ respectively. Thus, the overall loss function for the regressor composed of a summation of two loss function terms:

$${ℒ}_{R} = \frac{1}{2} [ {ℒ}_{R , 1} + {ℒ}_{R , 2} ] \qquad ( 10 )$$

And overall optimization loss is:

$${L}_{T} = α \cdot {L}_{R} + β \cdot {L}_{E} \qquad ( 11 )$$

Where α and β are hyper-parameters. The overall training process is presented in FIG. 5. Eventually model summed up in a form of:

$${y}_{i}^{\hat{}} = {f}_{θ} ( {x}_{i} , {p}_{i} ) = {R}_{ϕ} ( {E}_{φ} ( ( {x}_{i}^{-} , {p}_{i} ) , {p}_{i} ) , ϕ , φ ∈ θ \qquad ( 12 )$$

To assure that ƒ_{θ} is robust, we have used Online Hard Example Mining (OHEM) training strategy while minimizing L_{R} and L_{E}. After each loss was calculated per sample in batch, samples were sorted according to loss value in descending order. Bottom half of a loss were zeroed, such that gradient was calculated only for highest loss values. Utilized OHEM improves robustness of a network and prevents from over fitting to a specific sample behavior pattern. The proposed loss function in this process is:

$${ℒ}_{batch} = \frac{1}{N} {\sum }_{i = 1}^{N} {ℒ}_{i , sample} \qquad ( 13 )$$

$${ℒ}_{batch , OHEM} = \frac{1}{N} {\sum }_{i = 1}^{N} {w}_{i} \cdot {ℒ}_{i , sample} , {w}_{i} = \{ 0 , 1 \}$$

This enforcement will be used in all non-linear methods for fair comparison between them.

Following the original intention of a paper to implement prior knowledge of the world physical model acquired from the high-quality dataset to another model that works on low-quality data, we suggest a new transfer learning step. The engaging of high-quality knowledge is surprisingly simple. Due to Siamese setting, model is trained on two batches (or parts of batches) simultaneously. First of all, the new encoder E_{LQ,γ} with weights γ is initialized, then it replaces one of Siamese encoders. Instead of splitting batch, we apply two separate batches, each from different domain as described in FIG. 6. Apart from this change, process is same as initial training process. Note, that samples from different datasets are not correlated, and that the matching performed by contrastive loss, just as in single domain training.

For the sake of comparison, the proposed ER net-works compered to two other DNNs. A fully connected network (FC), and empirical estimated DNN architecture that is denoted as Regression module (R). Both FC and R correspond to straight forward approach described in (2.1). The custom networks (R and ER) networks composed mostly of cascade of convolutional layers and non-linear operations. Best results achieved with architectures presented in Table 5. GeLU activation and BatchNormalization were applied to most of the layers. FC parameters were chosen empirically. To date, most of chemometry models are linear, thus for the sake of completeness the NN results compered to a three linear regression methods OLS [XXXX], PLS [XXX], and SVR[XX].

TABLE 5

| | Layer | Input | | | Output |
|---|---|---|---|---|---|
| Function | Type | Dim. | Parameters | Activation | Dim. |
| Encoder | Conv. | BS, 221 + 1 | k = 4, s = 2, | GeLU | BS, 16, |
| | | | p = 0, d = 1 | | 111 |
| | Conv. | BS, 221 + 1 | k = 4, s = 2, | GeLU | BS, 16, |
| | | | p = 2, d = 2 | | 111 |
| | Conv. | BS, 221 + 1 | k = 4, s = 2, | GeLU | BS, 16, |
| | | | p = 5, d = 4 | | 111 |
| | Conv. | BS, 221 + 1 | k = 4, s = 2, | GeLU | BS, 16, |
| | | | p = 8, d = 6 | | 111 |
| | Conv. | BS, 64, 111 | k = 3, s = 2, | GeLU + BN | BS, 32, |
| | | | p = 0, d = 1 | | 55 |
| | Conv. | BS, 32, 55 | k = 3, s = 2, | GeLU + BN | BS, 32, |
| | | | p = 0, d = 1 | | 27 |
| | FC | BS, 32 · 27 | FC | — | BS, 2 |
| R | FC | BS, 2 + 1 | — | GeLU | BS, 64 |
| | Conv. | BS, 1, 64 | k = 8, s = 4, | GeLU + BN | BS, 24, |
| | | | p = 0, d = 1 | | 15 |
| | Conv. | BS, 24, 15 | k = 4, s = 3, | GeLU + BN | BS, 12, |
| | | | p = 0, d = 1 | | 7 |
| | FC | BS, 84 | — | — | BS, 1 |
| FC | FC | BS, 221 + 1 | — | GeLU | BS, 256 |
| | FC. | BS, 256 | — | GeLU | BS, 128 |
| | FC. | BS, 128 | — | GeLU | BS, 64 |
| | FC | BS, 64 | — | — | BS, 1 |

Network Architecture, where BS—Batch Size, BN—Batch Normalization. Layer's parameters: k—kernel size, s—stride, p—zero padding size and d—dilation.

The training was performed on a standardized data. Each sample compose of three elements (vi): spectrum, temperature and concentration. Each of which is standardized separately by Eq. 14:

$${v}_{i , norm} = \frac{vi - {V}^{-}}{σ v /} \qquad ( 14 )$$

Where V̅, and σ_{V} are the training set mean value and standard deviation respectively.

The training data is ordered in batches, which shuffled before every epoch. For the sake of over-fitting check, 10% of the training data was used for validation. In order to prevent misbalance and interfere to final result, the validation set selected uniformly across the whole data set. To achieve high quality encoding, namely, reduce dimension and separate samples by the corresponding parameters, both margins (for temperature and concentration) are based on same formula:

$${m}_{a , ij} = m \cdot ( \frac{\vert {a}_{i} - {a}_{j} \vert}{{a}_{ma x} - {a}_{m i n}} ) , a ∈ \{ c , T \} \qquad ( 15 )$$

Where m sets according to Eq. (7). OHEM was implemented for each loss and for each method. Batch size is set to 64, and training routine lasted 10,000 epochs under learning rate of 5·10⁻⁴ with applied weight decay of 10⁻³. Loss hyper parameters was α=7 and β=1 respectively.

Each of the DNN models (ER,R, and FC) was trained twenty times (M=20), i.e. twenty trained networks for each model. Evaluation preformed on N=290 test samples. Linear techniques were fitted under various parameters, and only best results are summarized. Quality of an estimation measured by few types of a sample error—absolute ε_{abs,i} and relative ε_{rel,i} as described below:

$${ε}_{ab s , i} = \vert {c}_{i} - {\hat{c}}_{i} \vert$$

$${ε}_{rel , i} = 100 \cdot \frac{{ε}_{a bs , i}}{{c}_{i}} , {c}_{i} \neq 0 \qquad ( 17 )$$

Overall quality of a models is measured by mean over all test set samples and iterations for both errors (Eq. 18, 19) and maximum relative error (Eq. 20). While motivation for former is straight forward, later quantifies accuracy in lower concentrations.

$${\overline{ε}}_{ab s} = \frac{1}{N \cdot M} {\sum }_{j = 1}^{M} [ {\sum }_{i = 1}^{N} {ε}_{ab s , i} ] \qquad ( 18 )$$

$${\overline{ε}}_{rel} = \frac{1}{N \cdot M} {\sum }_{j = 1}^{M} [ {\sum }_{i = 1}^{N} {ε}_{rel , i} ] \qquad ( 19 )$$

$${\overline{ε}}_{m ax} = \frac{1}{M} {\sum }_{j = 1}^{M} [ max \{ {ε}_{rel , i} \} \qquad ( 20 )$$

For completeness we present the R² score for each model.

Four scenarios were considered: (A) High-quality data with original dimension (d_{HQ}=221), (B) low-quality data (d_{LQ}=32), (C) low-quality data with simulated TLF and HLF peaks. Following above, the performance of the ER, R, and FC methods as well as previously suggested linear methods were investigated. The results of the various methods in the three scenarios are summarized in Table 6. Observing the results in all scenarios the DNN methods (FC, R, ER) out preformed the linear methods (OLS, PLS, SVR_{poly}). While the average relative error of the DNNs methods was ε̅ᵣₑₗ≥13.46% the average relative error of the linear methods was 433.41% which is unacceptable. As for the proposed DNN techniques, when models were trained on high-quality data results were similar, but the custom designed DNNs (R and ER) show slightly better performances than FC.

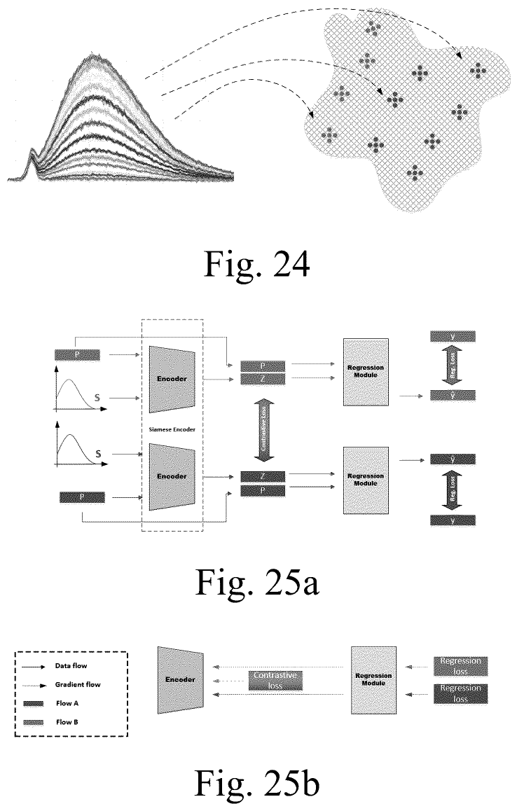

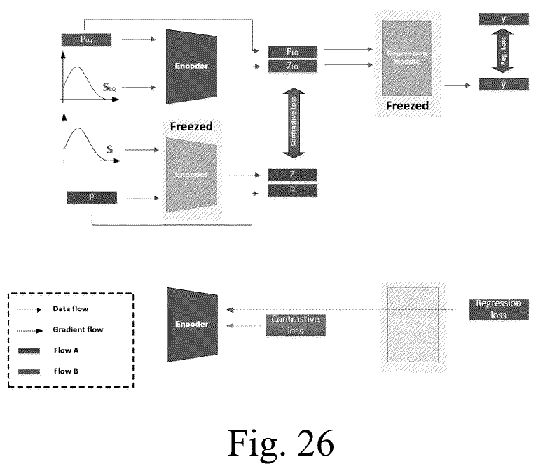

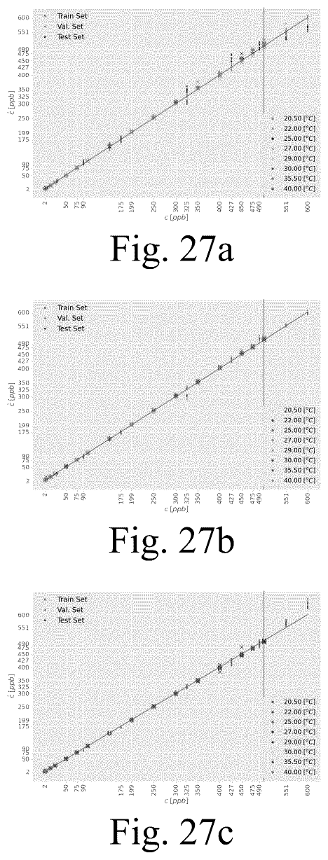

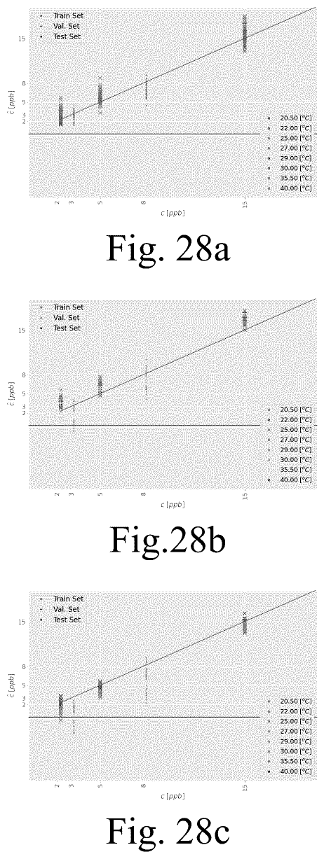

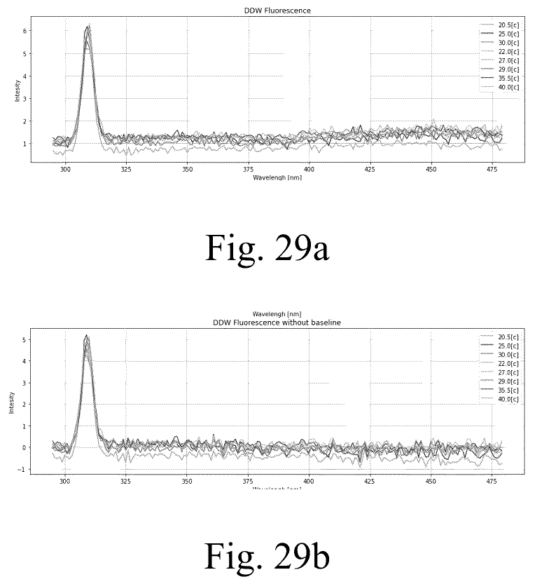

Regression curves of the DNNs performances for scenario A and scenario B are found at FIGS. 23 and 24 respectively. The structure of all sub figures is identical. The value of the real concentration is the x-axis, where the value of the predicted concentration is the y-axis. Each sub figure contains results from, training set, validation set, and test set. The equilibrium between the prediction to the real value resulted in the straight 45° line. Observing the FIG. 7, within the training range 227-500 ppb the results show a very good fitting quality between the prediction and the ground truth data for all the DNN models.

The interesting part is a comparison for low-quality data Scenarios B and C. While FC and R show lower performances due to the reduction in samples number from D_{HQ}=221 to d_{LQ}=32, the ER method show improvement in performance. The improvement resulted from the use of a prior knowledge that was achieved during training on high-quality data in training of the network for the low-quality data. This is especially important in the estimation of the low concentration where εₘₐₓ is showing half error than the R and FC networks resulted in ˜78% max relative error. While this error seems large, in a low ground truth concentration this resulted in only a 2.5 ppb error which is very low concentration to be measured by the means of fluorescence spectroscopy. Given a 500 ppb, this error reflects only 0.5% full scale error as a worst case scenario. A zoom in regression curves for low concentration up to 20 ppb for the DNN presented in FIG. 24.

TABLE 6
Comparison of regression methods for scenarios A, B and C.

| | | | | |
|---|---|---|---|---|
| Method | ε̅_̅{̅a̅b̅s̅}̅ [ppb] | ε̅ᵣ̅ₑ̅ₗ̅ [%] | εₘₐₓ [%] | R₂ |

Scenario A - Native data (d_{HQ} = 221)

| | | | | |
|---|---|---|---|---|
| OLS | 74.83 | 270.05 | 4045.44 | 0.79 |
| PLS | 29.89 | 184.36 | 2237.28 | 0.97 |
| SVR_{poly} | 87.56 | 385.73 | 2575.98 | 0.95 |
| FC | 6.94 | 14.62 | 184.63 | 0.99 |
| R | 6.67 | 10.06 | 113.8 | 0.99 |
| ER | 9.86 | 11.41 | 109.29 | 0.99 |

Scenario B - Decimated data (d_{LQ} = 32)

| | | | | |
|---|---|---|---|---|
| OLS | 77.28 | 449.94 | 5460.77 | 0.8 |
| PLS | 76.23 | 435.37 | 5247.87 | 0.8 |
| SVR_{poly} | 38.07 | 263.51 | 3204.61 | 0.95 |
| FC | 10.86 | 18.03 | 183.53 | 0.99 |
| R | 10.88 | 16.37 | 169.38 | 0.99 |
| ER | 9.13 | 9.28 | 78.97 | 0.99 |

Scenario C - Decimated and modified data (d_{LQ} = 32, HLFₚₑₐₖ)

| | | | | |
|---|---|---|---|---|
| OLS | 82.48 | 528.58 | 6918.52 | 0.16 |
| PLS | 166.37 | 1006.39 | 8944.64 | 0.16 |
| SVR_{rbf} | 67.48 | 376.73 | 6118.13 | 0.83 |
| FC | 11.40 | 15.60 | 158.3 | 0.99 |
| R | 12.08 | 16.44 | 149.02 | 0.99 |
| ER | 12.06 | 9.21 | 74.6 | 0.99 |

For regression task with both high-quality and low-quality data proposed method, just as trivial DNN solutions, achieves better results than linear ones. Difference is very noticeable—errors about tenth times lower with mean MAE˜10 [ppb] and relative error˜10 [%] in contrast to ˜30 [ppb] and ˜200 [%]. After clearing out that linear methods are no match to modern DNN approach, comparison shifts to nonlinear techniques and mainly ability to preserve prior knowledge from high-quality data where they have similar results. ER has a lead in low concentrations from a start with mean error of 3.46 [ppb], and surprisingly not only preserves its knowledge, but also manages to improve itself to 2.5 [ppb] with samples in low resolution and to 2.36 [ppb] with modified samples, which are as twice as better that other techniques. In addition, when quantity of data is reduced error rises with a delay compared to FC and R, therefore is also suggested for a scenarios with a low amount of low-quality train samples. Yet, the down side of ER, just as other methods, is in ability to cope with unbalanced data, thus this area is suggested for future work.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

Each set was prepared from the same stock solution of DDW and tryptophan with concentration of 10,000 part per billion [ppb] in a course of 3 days. Tryptophan solution was made from a solvent of Tryptophan powder (CAS number 73-22-3, Sigma-Aldrich, St. Louis, MO, USA). Samples preparation done by injecting small portions of stock solution to cuvette with previous tryptophan concentration solution, and thus increasing concentration. Fluorescence was measured by using RF-5301PC spectrofluorometric with magnetic stirrer cell holder (Shimadzu, Kyoto, Japan). Sample stirring done by using Multistirrer cc-301 magnetic stirrer (Scinics, Tokyo, Japan). Where measurements using standard 3.5 mL quartz cuvette (Hellma, Mullheim, Germany) having a path-length of 10 mm. Measurements kept in refrigerator in 4° C. The measurements in different temperatures achieved by heating up the samples first to room temperature (25° C.) and then to more than 40° C. using water bath 20B with thermostate VC/2 (Julabo, Seelbach, Germany) to worm up the cuvette. Upon measuring the fluorescence response the sample temperature was measured using TM-947SD thermometer with two T-type thermocouple probes (Sun—well Global, Dacun, Taiwan).

Measurements with the RF-5301PC were taken with excitation wavelength of 280 nm where Tryptophan is most efficient [35]. Illumination source was Xenon arc lamp, for the sake of monochromatic illumination (280 nm) and detailed spectral reading of emission in the 270-650 nm range detector's mono-chromators used 3 nm optical slits. Auto shutter was set to “On”, PMT (photo multiplier) set to high voltage mode. Baseline response was taken while the UV source was disable to insure consistency of the system through the experiment (FIG. 25) Eq. 27, where Δλ is wavelength range measurement, Sₘ and S_{b} are measured and base line spectra respectively.

$$S = {S}_{m} - {S}_{b} , S ∈ {R}^{Δ λ} \qquad ( 27 )$$

Following the above preparation step, sampling routine was performed as followed:
1. Cuvette is filled with 2200±20 μL of DDW was heated with Water bath to desired temperature. Various concentration and temperatures combinations accounted in Table 1.
2. Thermocouples are placed in measurement chamber and solution for temperature observation.
3. DDW fluorescence was scanned to assure clean baseline.
4. Measured part of the stock solution was injected to the cuvette to achieve the aimed concentration, while magnetic stirrer is on.
5. Waiting till temperature stabilization at specific point, with allowed error of +0.2° C.
6. Ten spectrum scans were taken for each sample. Outlier's filtration done on the fly by the operator.
7. Return to step four, until concentration reaches desired maximum.
8. Cuvette is cleaned and prepared for next series of samples.
Following sampling routine and Table 1, there were ten samples from each Concentration-Temperature (c-T) pair in the train set and in the test set. To allow real evaluation of the DNN's robustness training set and test sets were taken in different c-T sets with a test-train ratio of 0.36 (800 train samples and 290 test samples).

Tryptophan emission is typical to appear in range of [300,500] nm therefore fluorescence spectrum was limited to [280,500] nm to achieve 221 bins, which corresponds to resolution of 1 [nm/bin]. One of presented work's goals is to overcome unpredicted effect of equipment and environment on samples, therefore only easy accessed data from equipment was used that don't rely on steps or materials that inaccessible in field conditions.
No noise filtering was applied.
Concentration weren't recalculated according to base DDW volumes and thermal expansion for each series.
DDW Raman spectrum normalization was omitted.
Only fluorimeter's baseline was subtracted, due to its availability even in field conditions at every equipment.

Example of resulted series found in FIGS. 17a and 17b.

1. An inline early warning system of water contamination with fluorescence signature; said system comprising
a. an optical chamber embeddable into a water-supply system; said optical chamber having an internal passage configured for conducting a flow of water to be tested; said optical chamber having optically transparent entrance and exit windows;
b. a UV light source configured for illuminating said flow of water via said entrance window and excite tryptophan-like fluorescence at 280 nm and humic-like-fluorescence at 320-360 nm;
c. an optical sensor arrangement configured for sensing said tryptophan-like fluorescence and humic-like-fluorescence emissions at 350 nm and 400 to 480 nm via said exit window, respectively;
d. a UV light source configured for illuminating said flow of water via said entrance window and excite natural fluorescence of water contamination.
e. a non-optical sensor arrangement;
f. an acquisition and control unit configured for measuring tryptophan-like fluorescence and humic-like-fluorescence emissions and
g. a processor configured processing obtained measured data.

2. The inline early warning system according to claim 1 comprising a circulation jig for modelling detection of water contamination in outdoor conditions.

3. The inline early warning system according to claim 1, wherein said optical sensor arrangement comprises disclosed along to a propagation path of a fluorescence emission light beam an optical tube, a spectral dispersing element and an optical sensor.

4. The inline early warning system according to claim 1, wherein said exit window and optical sensor arrangement are optically connected by an optical fiber.

5. The inline early warning system according to claim 3, wherein said spectral filtering element static spectral filter, and lens, and interchangeable spectral filters.

6. The inline early warning system according to claim 3, wherein said spectral dispersing element is a diffraction grating.

7. The inline early warning system according to claim 3, wherein said optical sensor is an array of photoelectric multiplier tube.

8. The inline early warning system according to claim 3, wherein said non-optical sensor is a photoelectronic multiplier tube.

9. The inline early warning system according to claim 1, wherein said UV light source is a deep ultraviolet LED array.

10. The inline early warning system according to claim 1, wherein said arrangement comprises at least one sensor selected from the group consisting of a flow meter, a conductivity meter and thermocouple and any combination thereof.

11. The inline early warning system according to claim 1 comprising a deep neural network configured for single domain training with high-quality spectral data and transfer learning with low-quality spectral data.
